(19) United States
(12) Patent Application Publication
(10) Pub. No.: US 2009/0215796 A1
(43) Pub. Date: Aug. 27, 2009

(54) Compositions, Kits and Methods for a Titration Schedule for Pardoprunox Compounds

(75) Inventors: Juliana B. Bronzova, Weesp (NL); Gustaaf J. M. Van Scharrenburg, Weesp (NL); Serge V. Van De Witte, Weesp (NL); Hendrik Teunissen, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Solvay Pharmaceuticals B.V.

(21) Appl. No.: 12/391,393

(22) Filed: Feb. 24, 2009

Related U.S. Application Data
(60) Provisional application No. 61/031,134, filed on Feb. 25, 2008.
(60) Provisional application No. 61/079,558, filed on Jul. 10, 2008.

(51) Int. Cl. A61K 31/496; A61P 25/16; A61P 25/00

(52) U.S. Cl. 514/254.02

(57) ABSTRACT

The present invention is directed to compositions, kits, and methods for a titration schedule to facilitate the treatment of a central nervous system condition or disorder by administering a plurality of dosage units of a composition comprising a compound 7-(4-methyl)-1-piperazinyl-2(3H)-benzoxazolone (INN pardoprunox).

This application claims the benefit of U.S. provisional application No. 61/031,134, filed Feb. 25, 2008, and U.S. provisional application No. 61/079,558, filed Jul. 10, 2008, the disclosures of which are incorporated herein by reference.

The present invention relates to compositions, kits, and methods for a titration schedule to facilitate the treatment of a central nervous system (CNS) condition or disorder by administering a plurality of dosage units of a composition comprising a compound 7-(4-methyl)-1-piperazinyl-2(3H)-benzoxazolone hydrochloride (INNM pardoprunox hydrochloride) or a compound corresponding thereto, such as, its N-oxide and pharmaceutically acceptable salts, solvates and hydrates thereof, as well as solvates and hydrates of the salts.

The compound 7-(4-methyl)-1-piperazinyl-2(3H)-benzoxazolone (INN pardoprunox) has the following formula:

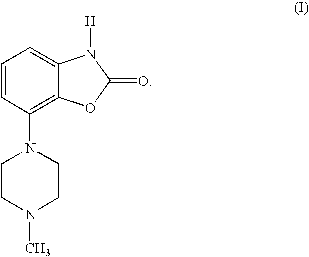

The hydrochloric acid salt of the above-referenced formula, i.e., 7-(4-methyl)-1-piperazinyl-2(3H)-benzoxazolone, is described and claimed in WO 00/029397. In addition, WO 05/107754 describes and claims the iontophoretic delivery of pardoprunox hydrochloride. Both WO00/029397 and WO05/107754 are hereby incorporated herein by reference.

Parkinson's disease (PD) is a neurodegenerative condition that produces progressive impairment in motor and non-motor functions. Current dopaminergic therapies target PD symptoms, but can also induce troublesome motor fluctuations and dyskinesias. These effects may be due to the pulsatile stimulation of dopamine receptors (Nutt et al., Trends Neurosci 2000; 23(10 Suppl): S109-S115; Olanow et al., Trends Neurosci 2000; 23(10 Suppl): S117-S126) and, therefore, the concept of ‘stabilizing’ the dopamine system by avoiding under- and over-stimulation, is an attractive prospect.

Partial dopamine agonists have shown agonistic action in brain regions with low dopamine tone and antagonistic action under conditions of high dopamine tone (McCreary et al., In: Ronken E, van Scharrenburg G J M, editors. Parkinson's disease (Solvay pharmaceutical conferences). USA: IOS Press; 2002:51-58; McDougall et al., Psychopharmacology 2005; 178:431-439; Koller and Herbster, Neurology 1987; 37(4):723-727). Therefore, these agents have the potential to provide full efficacy in PD during maintenance treatment, while avoiding the receptor ‘over-stimulation’ and associated adaptive changes that are characteristic of full agonists. Furthermore, avoiding excessive receptor stimulation may also reduce the occurrence of typical dopaminergic adverse events (AEs) such as dyskinesia (McCreary et al. In: Ronken E, van Scharrenburg G J M, editors. Parkinson's disease (Solvay Pharmaceuticals conferences). USA: IOS Press; 2002:51-58.). Although partial dopamine agonists have been investigated for the treatment of PD (Lieberman et al., Neurology 1987; 37(5): 863-865; Verhagen Metman et al., Mov Disord 1994; 9(5): 577-581; Ruggieri et al., Clin Neuropharmacol 1991; 14(5): 450-456), their clinical value is still under investigation.

Restless leg syndrome (RLS) is a sensorimotor disorder characterized by the uncontrollable urge to move the legs. This urge is accompanied by pain and unpleasant sensations and worsens often with rest (Martin, Consult Pharm. 2007, 22(11), 907-24). Treatment of RLS has the goal of alleviation of the primary symptoms of the disorder and the establishment of normal sleep. Dopamine agonists are considered the first line of treatment (Winkelman et al., Geriatrics 2007, 62(10), 13-6). However the dopamine agonist dose to treat RLS is much lower compared to doses used for the treatment of PD. Partial dopamine agonists effective in the treatment of PD are most likely also useful for the treatment of RLS at significant lower daily dosages compared to dosages necessary for the treatment of PD motorsymptoms. Gabapentin and opioids are of value for the treatment of refractory cases.

Pardoprunox (7-(4-methyl)-1-piperazinyl-2(3H)-benzoxazolone; also known as SLV308) is a partial dopamine D₂ and D₃ receptor agonist that also has full 5-HT_{1A} agonist activity (Glennon et al., Synapse 2006; 60: 599-608). Studies using animal models have indicated that pardoprunox has a marked and long-lasting anti-PD effect, with evidence of antidepressant and anxiolytic efficacy (McCreary et al., In: Ronken E, van Scharrenburg G J M, editors. Parkinson's disease (Solvay Pharmaceuticals conferences). USA: IOS Press; 2002: 51-58; Johnston et al., Mov Disord 2005; 20(S10): P16: Poster). As such, it was prudent to test the hypothesis that pardoprunox is efficacious for the treatment of PD, as it is relatively well tolerated in healthy human subjects.

As indicated above, during clinical investigations on pardoprunox hydrochloride, the compound was well tolerated; however, undesirable adverse events such as nausea, vomiting and orthostatic hypertension can occur during the initiation of treatment, which may in some instances lead to early discontinuance of the treatment and/or noncompliance with the treatment plan. As such, there is a need to not only reduce these undesired side effects at the initiation of treatment, but also ensure compliance with the treatment plan. It has now been surprisingly discovered that at least one of these undesired side effects can be prevented by starting at a very low dose level compared with the maintenance level and by a gradual increase of the pardoprunox compound dose according to a specific titration schedule.

As used herein, the term “pardoprunox compound(s)” refers to the active compound 7-(4-methyl)-1-piperazinyl-2(3H)-benzoxazolone, its N-oxide and pharmaceutically acceptable salts, solvates and hydrates thereof, as well as solvates and hydrates of the salts. By the expression “at least one pardoprunox compound” in the framework of the present invention, the inventors include one of the active compounds mentioned above or a mixture of one or more of the active compounds mentioned above. Pharmaceutically acceptable salts of pardoprunox or its N-oxide may be obtained using standard procedures well known in the art, for example, by mixing a compound of the present invention with a suitable acid, for instance an inorganic acid or an organic acid.

Accordingly, the present invention is directed to compositions, kits, and methods using a titration schedule to facilitate the treatment of a CNS condition or disorder by administering a plurality of dosage units comprising a pardoprunox compound such as the compound 7-(4-methyl)-1-piperazinyl-2(3H)-benzoxazolone hydrochloride. For example, the present invention is related to a composition regimen for a titration schedule to facilitate the treatment of a central nervous system condition comprising a plurality of unit dosages of a composition, each of the unit dosages comprising at least one pardoprunox compound, wherein the unit dosages over the entire titration schedule increase in amount of the at least one pardoprunox compound.

Further disclosed herein is a kit for a titration schedule to facilitate the treatment of a central nervous system condition comprising a plurality of unit dosages of a composition comprising at least one pardoprunox compound, wherein the unit dosages over the entire titration schedule increase in amount of the at least one pardoprunox compound.

In addition, the present invention is related to a method using a titration schedule for the treatment of a central nervous system condition in a subject in need thereof comprising administering to the subject a composition regimen comprising a plurality of unit dosages of the composition, each of the unit dosages comprises at least one pardoprunox compound, wherein the unit dosages over the entire titration schedule increase in amount of the at least one pardoprunox compound. Finally, disclosed herein is a method for reducing at least one side effect associated with the initiation of a pardoprunox treatment, such as nausea, vomiting and orthostatic hypotension, comprising administering a composition regimen comprising a plurality of unit dosages of a composition, each of the unit dosages comprises at least one pardoprunox compound according, wherein the unit dosages over the titration schedule increase in amount of the at least one pardoprunox compound.

As used herein, the term “titration schedule” refers to a regimen of dosages of a pharmaceutically active agent over a period of time, based in part on a target dose and/or a maintenance dose.

Pardoprunox compounds are indicated for the treatment of CNS disorders such as mood disorders, Restless Leg Syndrome and Parkinson's disease. In the framework of the present invention dosage strengths are expressed in an amount equivalent to pardoprunox base. As used herein, the term “pardoprunox base” refers to the compound 7-(4-methyl)-1-piperazinyl-2(3H)-benzoxazolone (INN pardoprunox) having the following formula:

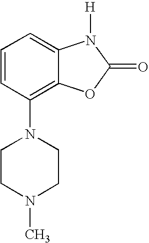

As used herein the term strength refers to the amount of the active ingredient present in the dose. The typical dosing regimen ranges from amounts equivalent to 0.1 mg to 12 mg pardoprunox base per day but higher doses such as doses up to amounts equivalent to 42 mg pardoprunox base per day may be given; doses may vary based in part on the severity of the CNS condition and other conditions of the patient.

For example, a dose for pardoprunox compounds (the target dose and/or maintenance dose) can be a dose equivalent to 0.3 mg/day, 0.6 mg/day, 0.9 mg/day, 1.2 mg/day, 1.5 mg/day, 3.0 mg/day 4.5 mg/day, 6.0 mg/day, 9.0 mg/day, 12.0 mg/day, 15.0 mg/day, 21.0 mg/day, 24.0 mg/day, 27.0 mg/day, 30.0 mg/day, 36.0 mg/day or 42.0 mg/day of pardoprunox base. Daily doses are achieved by dosing once, twice or three times on the same day of the same unit dosage or different unit dosages. In clinical studies, the efficacy, safety and tolerability profile of pardoprunox suggests it may be an effective agent to employ as a treatment option for patients with early and advanced Parkinson's disease and Restless Leg Syndrome. Despite the efficacy, safety and tolerability profiles, pardoprunox treatment can result in adverse events during initiation of treatment, which may lead to the discontinuation of pardoprunox treatment and/or inconsistent use of the treatment.

As used herein the term unit dosage refers to a single separate unit containing the active ingredient in a certain amount, such as a tablet or a capsule.

The pardoprunox compound administered in the framework of the titration schedule, in the form of a plurality of unit dosages of a composition, can be over the course of a period chosen from, for example, 3 to 84 consecutive days, such as 3 to 14 days or 3 to 28 days, and further for example, from 3 to 49 days. Alternatively, the period of time can be divided into time segments other than one day, such as two days, three days, four days, five days, six days or even seven days. In that case, the same unit dosage of pardoprunox compound can be administered every day within the time segment. The titration schedule can also comprise time segments of different duration, for example, a combination of time segments of 1 day, 2 days, 3 days, 4 days, 5 days, 6 days and 7 days. The number of time segments ranges between three and twenty and can therefore, for example, also be four, five, six, seven, eight, ten, twelve or fifteen. The period over which the titration schedule spans may be in part due to the target and/or maintenance dose; for example, a titration schedule may span over a larger number of days (such as 56 days) due to the elevated target and/or maintenance dose, or it may span over a shorter period of time (such as 3 days) with a lower target and/or maintenance dose depending on the disease and condition and/or age and/or gender of the patient.

The strength or amount of pardoprunox compound in each unit dosage increases incrementally over the subsequent titration schedule until a target dose and/or maintenance dose is reached. Alternatively, consecutive strengths or amounts of unit dosages may be given during the course of the titration such that over the entire titration schedule, an overall increase in strength or amount of the unit dosage results. For example, comparing the first unit dose to the last unit dose before the maintenance unit dosage is administered, there is an increase in the amount of pardoprunox compound being administered. A first low dose to start the titration regimen with is e.g a dose equivalent with an amount of 0.1 or 0.3 mg/day pardoprunox base, divided over 1, 2 or 3 unit doses. In the framework of the present invention all indicated doses or strength are based on the administration of 3 unit dosages per day. In case the daily dose of pardoprunox compound is divided over 2 unit dosages the indicated doses have to be multiplied by a factor 1.5; when the daily dose of pardoprunox compound is given as a single dosage unit the indicated doses have to be multiplied by a factor 3. In some embodiments the titration regimen starts with a single administration of the lowest dose on the first day (indicated as day 0).

In one embodiment, each of the unit dosages of the composition are chosen from single strength doses equivalent to pardoprunox base of from about 0.025 mg to about 0.075 mg, from about 0.075 mg to about 0.15 mg, from about 0.15 mg to about 0.25 mg, from about 0.25 mg to about 0.35 mg, from about 0.35 mg to about 0.45 mg, from about 0.45 mg to about 0.55 mg, from about 0.55 mg to about 0.65 mg, from about 0.65 mg to about 0.8 mg, from about 0.8 mg to about 1.2 mg, from about 1.2 mg to about 1.7 mg, from about 1.7 mg to about 2.2 mg from about 2.2 mg to about 2.7 mg, from about 2.7 mg to about 3.5 mg, from about 3.5 mg to about 4.5 mg, from about 4.5 mg to about 5.5 mg, from about 5.5 mg to about 6.5 mg, from about 6.5 mg to about 7.5 mg, from about 7.5 mg to about 8.5 mg, from about 8.5 mg to about 9.5 mg, from about 9.5 mg to about 11.0 mg, and from about 11.0 mg to about 13.0 mg. When choosing the different strengths of each unit dosage, the strength can be the subsequent strength within the group indicated above.

In a further embodiment, each of the unit dosages of the compositions are chosen from single strength doses equivalent to pardoprunox base of from about 0.04 mg to about 0.06 mg, from about 0.08 mg to about 0.12 mg, from about 0.18 mg to about 0.22 mg, from about 0.28 mg to about 0.32 mg, from about 0.38 mg to about 0.42 mg, from about 0.48 mg to about 0.52 mg, from about 0.58 mg to about 0.62 mg, from about 0.68 mg to about 0.72 mg, from about 0.78 mg to about 0.82 mg, from about 0.88 mg to about 0.92 mg, from about 0.98 mg to about 1.1 mg, from about 1.3 to about 1.7 mg, from about 1.8 mg to about 2.2 mg, from about 2.3 mg to about 2.7 mg, from about 2.8 mg to about 3.2 mg, from about 3.8 to about 4.2 mg, from about 4.8 to about 5.2 mg, from about 5.8 to about 6.2 mg, from about 6.8 mg to about 7.2 mg, from about 7.8 mg to about 8.2 mg, from about 8.8 mg to about 9.2 mg, from about 9.8 mg to about 10.4 mg, and from about 11.6 mg to about 12.4 mg of a pardoprunox compound. When choosing the different strengths of each unit dosage, the strength can be the subsequent strength within the group indicated above.

In yet a further embodiment of the titration schedule, the strength or amount of each unit dosage may be chosen from single strength doses equivalent to pardoprunox base of about 0.05 mg, about 0.1 mg, about 0.2 mg, about 0.3 mg, about 0.4 mg, about 0.5 mg, about 0.6 mg, about 0.7 mg, about 1.0 mg, about 1.5 mg, about 2.0 mg, about 2.5 mg, about 3.0 mg, about 4.0 mg, about 5.0 mg, about 6.0 mg, about 7.0 mg, about 8.0 mg, about 9.0 mg, about 10.0 mg, and about 12.0 mg.

Within the titration schedule, the unit dosages may increase in an amount or strength of the pardoprunox compound ranging from about 1.1 to 3 times that of the preceding dose and further for example, from about 1.5 to 2.5 times that of the preceding dose. In the case of the first dose of the titration schedule, there is no preceding dose, but with the remaining dosages of the titration schedule, a preceding dosage is available for consideration. When necessary the titration dose can also be provided in the form of a combination of single unit dosages to give the desired total strength.

The strengths of the unit dosages indicated in par [018] to par [022] above are based on the administration of the unit dosages three times a day. When the unit dosages are administered two times a day said strengths are multiplied by a factor 1.5 and when the unit dosages are administered once daily said strength are multiplied by a factor three.

The composition regimen provided in the present disclosure can be in the form of a kit comprising, e.g., a plurality of unit dosages in the form of tablets for the titration schedule to arrive at the final and/or maintenance dose. The present invention is further directed to a kit for a titration schedule to facilitate the treatment of a central nervous system condition comprising a plurality of unit dosages of a composition comprising at least one pardoprunox compound, wherein the unit dosages over the titration schedule increase in amount of the pardoprunox compound. The strengths or amounts of each unit dosage can be chosen from the groups of different strengths indicated above. In general, titration schedules used to arrive at final maintenance dosages selected for the treatment of PD symptoms will be much longer compared to the titration schedules to get to maintenance dosages for the treatment of RLS. This is a consequence of the fact that treatment of RLS requires significantly lower daily maintenance dosages of a certain dopamine agonist compared to efficacious daily maintenance dosages for PD of the same dopamine agonist. A titration schedule for RLS will contain at least 3 unit dosage when the pardoprunox compound is administered once daily, 6 unit dosage when the pardoprunox compound is administered twice daily and 9 unit dosage when the pardoprunox compound is administered three times daily.

In one embodiment the kit comprises 6 unit dosages with a strength equivalent to pardoprunox base of about 0.08 mg to about 0.12 mg, 6 unit dosages with a strength equivalent to pardoprunox base of from about 0.18 mg to about 0.22 mg, 9 unit dosages with a strength equivalent to pardoprunox base of from about 0.28 mg to about 0.32 mg, 9 unit dosages with a strength equivalent to pardoprunox base of from about 0.38 mg to about 0.42 mg, 12 unit dosages with a strength equivalent to pardoprunox base of from about 0.48 mg to about 0.52 mg, 9 unit dosages with a strength equivalent to pardoprunox base of from about 0.68 mg to about 0.72 mg, 12 unit dosages with a strength equivalent to pardoprunox base of from about 0.9 mg to about 1.1 mg, 21 unit dosages with a strength equivalent to pardoprunox base of about 1.4 mg to about 1.6 mg and 21 unit dosages with a strength equivalent to pardoprunox base of from about 1.9 mg to about 2.1 mg. Optionally the kit also contains an additional unit dosage with a strength equivalent to pardoprunox base of about 0.08 mg to about 0.12 mg or three unit dosages with a strength equivalent to pardoprunox base of about 0.04 to about 0.06 mg.

In a more specific embodiment the kit comprises 6 unit dosages with a strength equivalent to pardoprunox base of about 0.1 mg, 6 unit dosages with a strength equivalent to pardoprunox base of about 0.2 mg, 9 unit dosages with a strength equivalent to pardoprunox base of about 0.3 mg, 9 unit dosages with a strength equivalent to pardoprunox base of about 0.4 mg, 12 unit dosages with a strength equivalent to pardoprunox base of about 0.5 mg, 9 unit dosages with a strength equivalent to pardoprunox base of about 0.7 mg, 12 unit dosages with a strength equivalent to pardoprunox base of about 1.0 mg, 21 unit dosages with a strength equivalent to pardoprunox base of about 1.5 mg and 21 unit dosages with a strength equivalent to pardoprunox base of about 2.0 mg of a pardoprunox compound. Optionally the kit also contains an additional unit dosage with a strength equivalent to pardoprunox base of about 0.1 mg or three unit dosages with a strength equivalent to pardoprunox base of about 0.05 mg.

In a further embodiment the kit comprises 6 unit dosages with a strength equivalent to pardoprunox base of 0.08 mg to about 0.12 mg, 6 unit dosages with a strength equivalent to pardoprunox base of from about 0.18 mg to about 0.22 mg, 9 unit dosages with a strength equivalent to pardoprunox base of from about 0.28 mg to about 0.32 mg, 21 unit dosages with a strength equivalent to pardoprunox base of from about 0.48 mg to about 0.52 mg and 21 unit dosages with a strength equivalent to pardoprunox base of from about 0.98 mg to about 1.1 mg, 21 unit dosages with a strength equivalent to pardoprunox base of about 1.4 mg to about 1.6 mg and 21 unit dosages with a strength equivalent to pardoprunox base of from about 1.9 mg to about 2.1 mg of a pardoprunox compound. Optionally the kit also contains an additional unit dosage with a strength equivalent to pardoprunox base of about 0.08 mg to about 0.12 mg or three unit dosages with a strength equivalent to pardoprunox base of about 0.04 to about 0.06 mg.

In a further more specific embodiment the kit comprises 6 unit dosages with a strength equivalent to pardoprunox base of about 0.1 mg, 6 unit dosages with a strength equivalent to pardoprunox base of about 0.2 mg, 9 unit dosages with a strength equivalent to pardoprunox base of about 0.3 mg, 21 unit dosages with a strength equivalent to pardoprunox base of about 0.5 mg, 21 unit dosages with a strength equivalent to pardoprunox base of about 1.0 mg, 21 unit dosages with a strength equivalent to pardoprunox base of about 1.5 mg and 21 unit dosages with a strength equivalent to pardoprunox base of about 2.0 mg, of a pardoprunox compound. Optionally the kit also contains an additional unit dosage with a strength equivalent to pardoprunox base of about 0.1 mg or three unit dosages with a strength equivalent to pardoprunox base of about 0.05 mg.

In a further embodiment the kit comprises 6 unit dosages with a strength equivalent to pardoprunox base of 0.08 mg to about 0.12 mg, 6 unit dosages with a strength equivalent to pardoprunox base of from about 0.18 mg to about 0.22 mg, 9 unit dosages with a strength equivalent to pardoprunox base of from about 0.28 mg to about 0.32 mg and 21 unit dosages with a strength equivalent to pardoprunox base of from about 0.48 mg to about 0.52 mg of a pardoprunox compound. Optionally the kit also contains an additional unit dosage with a strength equivalent to pardoprunox base of about 0.08 mg to about 0.12 mg or three unit dosages with a strength equivalent to pardoprunox base of about 0.04 to about 0.06 mg.

In a further more specific embodiment the kit comprises 6 unit dosages with a strength equivalent to pardoprunox base of about 0.1 mg, 6 unit dosages with a strength equivalent to pardoprunox base of about 0.2 mg, 9 unit dosages with a strength equivalent to pardoprunox base of about 0.3 mg and 21 unit dosages with a strength equivalent to pardoprunox base of about 0.5 mg of a pardoprunox compound. Optionally the kit also contains an additional unit dosage with a strength equivalent to pardoprunox base of about 0.1 mg or three unit dosages with a strength equivalent to pardoprunox base of about 0.05 mg.

In a further embodiment the kit comprises 18 unit dosages with a strength equivalent to pardoprunox base of from about 0.08 mg to about 0.12 mg, 18 unit dosages with a strength equivalent to pardoprunox base of from about 0.15 mg to about 0.25 mg, 18 unit dosages with a strength equivalent to pardoprunox base of from about 0.25 mg to about 0.35 mg, 18 unit dosages with a strength equivalent to pardoprunox base of from about 0.35 mg to about 0.45 mg, and optionally 18 unit dosages with a strength equivalent to pardoprunox base of from about 0.45 mg to about 0.55 mg, 18 unit dosages with a strength equivalent to pardoprunox base of from about 0.55 mg to about 0.8 mg, 18 unit dosages with a strength equivalent to pardoprunox base of from about 0.8 mg to about 1.2 mg and 18 unit dosages with a strength equivalent to pardoprunox base of from about 1.2 mg to about 1.8 mg. Optionally the kit also contains an additional unit dosage with a strength equivalent to pardoprunox base of about 0.08 mg to about 0.12 mg or three unit dosages with a strength equivalent to pardoprunox base of about 0.04 to about 0.06 mg.

In a further more specific embodiment the kit comprises 18 unit dosages with a strength equivalent to pardoprunox base of about 0.1 mg, 18 unit dosages with a strength equivalent to pardoprunox base of about 0.2 mg, 18 unit dosages with a strength equivalent to pardoprunox base of about 0.3 mg, 18 unit dosages with a strength equivalent to pardoprunox base of about 0.4 mg, and optionally 18 unit dosages with a strength equivalent to pardoprunox base of about 0.5 mg, 18 unit dosages with a strength equivalent to pardoprunox base of about 0.7 mg, 18 unit dosages with a strength equivalent to pardoprunox base of about 1.0 mg and 18 unit dosages with a strength equivalent to pardoprunox base of about 1.5 mg of a pardoprunox compound. Optionally the kit also contains an additional unit dosage with a strength equivalent to pardoprunox base of about 0.1 mg or three unit dosages with a strength equivalent to pardoprunox base of about 0.05 mg.

In a further embodiment the kit comprises 12 unit dosages with a strength equivalent to pardoprunox base of from about 0.08 mg to about 0.12 mg, 12 unit dosages with a strength equivalent to pardoprunox base of from about 0.15 mg to about 0.25 mg, 12 unit dosages with a strength equivalent to pardoprunox base of from about 0.25 mg to about 0.35 mg, 12 unit dosages with a strength equivalent to pardoprunox base of from about 0.35 mg to about 0.45 mg, and optionally 12 unit dosages with a strength equivalent to pardoprunox base of from about 0.45 mg to about 0.55 mg, 12 unit dosages with a strength equivalent to pardoprunox base of from about 0.55 mg to about 0.8 mg, 12 unit dosages with a strength equivalent to pardoprunox base of from about 0.8 mg to about 1.2 mg and 12 unit dosages with a strength equivalent to pardoprunox base of from about 1.2 mg to about 1.8 mg. Optionally the kit also contains an additional unit dosage with a strength equivalent to pardoprunox base of about 0.08 mg to about 0.12 mg or three unit dosages with a strength equivalent to pardoprunox base of about 0.04 to about 0.06 mg.

In a further more specific embodiment the kit comprises 12 unit dosages with a strength equivalent to pardoprunox base of about 0.1 mg, 12 unit dosages with a strength equivalent to pardoprunox base of about 0.2 mg, 12 unit dosages with a strength equivalent to pardoprunox base of about 0.3 mg, 12 unit dosages with a strength equivalent to pardoprunox base of about 0.4 mg, and optionally 12 unit dosages with a strength equivalent to pardoprunox base of about 0.5 mg, 12 unit dosages with a strength equivalent to pardoprunox base of about 0.7 mg, 12 unit dosages with a strength equivalent to pardoprunox base of about 1.0 mg and 12 unit dosages with a strength equivalent to pardoprunox base of about 1.5 mg of a pardoprunox compound. Optionally the kit also contains an additional unit dosage with a strength equivalent to pardoprunox base of about 0.1 mg or three unit dosages with a strength equivalent to pardoprunox base of about 0.05 mg.

In another embodiment the kit comprises at least three compositions containing increasing amounts of a pardoprunox compound and in an even further embodiment the kit comprises at least seven compositions containing increasing amounts of a pardoprunox compound.

The strengths of the unit dosages in the kits indicated in par [024] to par [036] above are based on the administration of the unit dosages three times a day. When the unit dosages are administered two times a day a kit is provided wherein said strengths are multiplied by a factor of 1.5 and when the unit dosages are administered once daily a kit is provided, wherein said strengths are multiplied by a factor of three.

The kit as described above can be in the form of a package, such as a blister package or dispenser, the package comprising a plurality of tablets, organized in an order which enables the required titration schedule up to and optionally including the maintenance dose and further for example, having indicia disposed adjacent to the tablets for displaying successive strengths and/or successive days.

In further embodiments, the kit can be in the form a package comprising a plurality of capsules, granular aerosols, suppositories and/or suspensions to form each unit dosage. Such dosage forms can be prepared by mixing, individually or together, the pardoprunox compound with inert pharmaceutically acceptable excipients, carriers and/or pharmaceutically acceptable ingredients.

In the preparation of the compositions of the present disclosure, the active ingredients, i.e., at least one pardoprunox compound, may be mixed with solid, powdered ingredients, such as lactose, saccharose, sorbitol, mannitol, starch, amylopectin, cellulose derivatives, gelatin, or another suitable ingredient, as well as with binders, disintegrating agents and lubricating agents such as povidone, crospovidone, magnesium stearate, calcium stearate, sodium stearyl fumarate and polyethylene glycol waxes. The mixture may then be processed into granules, pressed into tablets, and/or any other known pharmaceutical form such as suppositories and/or suspensions.

Soft gelatin capsules may further be prepared containing a composition comprising a mixture of the active ingredients of the invention, vegetable oil, fat, or other suitable vehicle for soft gelatin capsules. Hard gelatin capsules may contain granules of the active ingredients. Hard gelatin capsules may also contain the active ingredients in combination with solid powdered ingredients such as lactose, saccharose, sorbitol, mannitol, potato starch, corn starch, amylopectin, cellulose derivatives or gelatin.

In addition, compositions of the present disclosure can comprise at least one pharmaceutical excipient. Non-limiting examples of suitable excipients include suspending agents (for example, gums, xanthans, cellulosics and sugars), humectants (for example, sorbitol), solubilizers (for example, ethanol, water, PEG and propylene glycol), surfactants (for example, sodium lauryl sulfate, Spans, Tweens, and cetyl pyridine), preservatives, antioxidants (for example, parabens, and vitamins E and C), anti-caking agents, coating agents, chelating agents (for example, EDTA), stabilizers, antimicrobial agents, antifungal or antibacterial agents (for example, parabens, chlorobutanol, phenol, sorbic acid), isotonic agents (for example, sugar, sodium chloride), thickening agents (for example, methyl cellulose), flavoring agents (for example, chocolate, thalmantin, aspartame, root beer or watermelon or other flavorings stable at pH 7 to 9), anti-foaming agents (e.g., simethicone, Mylicon®), disintegrants, flow aids, lubricants, adjuvants, colorants, diluents, moistening agents, preservatives, carriers, binders (for example, hydroxypropylmethylcellulose, polyvinyl pyrrolidone, other cellulosic materials and starch), diluents (for example, lactose and other sugars, starch, dicalcium phosphate and cellulosic materials), disintegrating agents (for example, starch polymers and cellulosic materials), glidants (for example, colloidal silica) and water insoluble or water soluble lubricants or lubricating agents.

The active ingredients may be separately premixed with the non-active ingredients, before being mixed to form a formulation. The active ingredients may also be mixed with each other, before being mixed with the non-active ingredients to form a formulation.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification, including the examples and attached claims, are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result.

### EXAMPLES

### Example 1

### Materials and Methods

Pardoprunox hydrochloride (7-(4-methyl-1-piperazinyl)-2(3H)-benzoxazolone hydrochloric acid salt) can be synthesized as described in WO00/29397 and Drugs of the Future 2001, 26, 128-32. Microcrystalline cellulose, povidone, crospovidone, colloidal anhydrous silica, sodium stearyl fumarate, microcrystalline cellulose and Opadry® were used in pharmaceutical grades available from common commercial sources.

### Example 2

### Preparation of Tablets of Different Strength

Tablets with a strength equivalent to 0.1 to 10 mg of pardoprunox base per tablet were prepared according to the following procedures (required quantities for all strengths are given in Table 1 and 2 below):

The granulation liquid was made by dissolving the pardoprunox hydrochloride in 75-80% of the required water quantity.

The required quantities of microcrystalline cellulose, povidone and crospovidone were mixed.

The granulation liquid and the remainder of the water was added to the mixture; the mixture was granulated.

After drying, the granulate was screened using a 1 mm rasp resulting in a good blend uniformity.

The required quantities of colloidal anhydrous silica and sodium stearyl fumarate were added and mixed with the powder mixture.

This final powder mixture was compressed into tablets with a core weight of 240 mg.

TABLE 1

| | 0.1 mg | 0.2 mg | 0.3 mg | 0.4 mg | 0.5 mg | 0.7 mg | |
|---|---|---|---|---|---|---|---|
| | tablet | tablet | tablet | tablet | tablet | tablet | |
| Component | Quantity (kg) | | | | | | Ref¹ |
| pardoprunox hydrochloride | 0.01445 | 0.02891 | 0.04336 | 0.05781 | 0.07228 | 0.1012 | |
| Cellulose, microcrystalline | 28.000 | 27.990 | 27.980 | 27.960 | 27.950 | 27.930 | Ph. Eur. |
| (Avicel PH102 ®) | | | | | | | |
| Povidone (Kollidon 25 ®) | 1.350 | 1.350 | 1.350 | 1.350 | 1.350 | 1.350 | Ph. Eur. |
| Crospovidone | 0.300 | 0.300 | 0.300 | 0.300 | 0.300 | 0.300 | Ph. Eur. |
| Silica, colloidal anhydrous | 0.0300 | 0.0300 | 0.0300 | 0.0300 | 0.0300 | 0.0300 | Ph. Eur. |
| (Aerosil 200 Pharma ®) | | | | | | | |
| Sodium stearyl fumarate | 0.300 | 0.300 | 0.300 | 0.300 | 0.300 | 0.300 | Ph. Eur. |
| Water, purified | 11.500 | 11.500 | 11.500 | 11.500 | 11.500 | 11.500 | Ph. Eur. |

¹If Ph. Eur. quality is not available, USP/NF quality may be used

TABLE 2

| | 1.0 mg | 1.5 mg | 2.0 mg | 4.0 mg | 6.0 mg | 8.0 mg | 10 mg | |
|---|---|---|---|---|---|---|---|---|
| | tablet | tablet | tablet | tablet | tablet | tablet | tablet | |
| Component | Quantity (kg) | | | | | | | Ref¹ |
| pardoprunox hydrochloride | 0.1445 | 0.2169 | 0.2891 | 0.5781 | 0.8673 | 1.156 | 1.445 | |
| Cellulose, microcrystalline | 27.880 | 27.800 | 27.730 | 27.440 | 27.150 | 26.860 | 26.580 | Ph. Eur. |
| (Avicel PH102 ®) | | | | | | | | |
| Povidone (Kollidon 25 ®) | 1.350 | 1.350 | 1.350 | 1.350 | 1.350 | 1.350 | 1.350 | Ph. Eur. |
| Crospovidone | 0.300 | 0.300 | 0.300 | 0.300 | 0.300 | 0.300 | 0.300 | Ph. Eur. |
| Silica, colloidal anhydrous | 0.0300 | 0.0300 | 0.0300 | 0.0300 | 0.0300 | 0.0300 | 0.0300 | Ph. Eur. |
| (Aerosil 200 Pharma ®) | | | | | | | | |
| Sodium stearyl fumarate | 0.300 | 0.300 | 0.300 | 0.300 | 0.300 | 0.300 | 0.300 | Ph. Eur. |
| Water, purified | 11.500 | 11.500 | 11.500 | 11.500 | 11.500 | 11.500 | 11.500 | Ph. Eur. |

¹If Ph. Eur. quality is not available, USP/NF quality may be used

A suspension of the required Opadry® coating material in purified water was prepared. The tablets were coated by spraying with the Opadry® suspension, to about 3.5% of the tablet core weight. The product was packed and tested.

All tablet strengths were prepared in a corresponding manner by decreasing or increasing the amount of pardoprunox hydrochloride compensating with the amount of microcrystalline cellulose to come to the same final weight, with the understanding that a different coating component was used in order to obtain a different color for every tablet strength (see Table 1 and 2). All other strengths, including the strengths of 0.05 mg, 0.15 mg, 0.6 mg, 0.9 mg, 1.2 mg, 2.1 mg, 3.0 mg, 4.5 mg, 7.5 mg and 12.0 mg can be prepared in the same way.

### Example 3

### Clinical Study

In different clinical studies different titration schedules were used.

In a double-blind, placebo-controlled proof of principle (PoP) study (S308.2.004), the effects of pardoprunox on efficacy, safety and tolerability as monotherapy in early PD were investigated including 138 randomized and treated subjects. Following a fixed-flexible titration period (average length of 24 days), subjects were kept on maintenance dose levels for three weeks (9, 12, 15, 18, 24, 30, 36 or 45 mg/day). Study medication was taken orally three times daily (tid), in the morning, at noon and in the evening, with any missed doses not taken subsequently.

The dose of pardoprunox was progressively titrated from 0.3 mg/day to 9 mg/day according to a predefined schedule within 10 days, and to 30 mg/day within 20 days, by giving single dose unit or a combination of single dose unit. Daily doses were given over the day in the form of three dosage units. Up to the dose of 30 mg/day the titration was based on tolerability only, meaning that every next day (until step 10) or every other day (step 10-14) the dose was to be increased with one step if well-tolerated by the patient (see Table 3). If a patient did not tolerate the daily dose due to adverse events, the patient could be instructed to decrease the dose to the previous level (one titration step down). From that moment, the patient had to stay on that lower dose level for 2 full days (at least 6 administrations) before trying the higher level again. The first administration of the higher dose had to be done in the morning. For a given titration step only one subsequent second attempt was allowed to reach the higher dose level. In total, three dose adjustments (on different titration steps) were allowed. If patients still could not reach a certain dose level after two sequential attempts and that dose level was below the minimum required dose level of 9 mg/day, they were not allowed to enter the maintenance phase. If a dose level of at least 9 mg/day has already been reached, they were allowed to maintain that dose level for the entire maintenance phase. Patients who reached the 30 mg/day dose could continue to escalate to 45 mg if tolerability allowed, and if further efficacy was expected. The total duration of the titration phase was 2-6 weeks, depending on the number of dose reductions needed. For all patients who reached an individual best maintenance dose in the predefined range of 9-45 mg/day the treatment was continued for 3 weeks. This was followed by 2-4 days of down-titration and discontinuation during a 1-week follow-up period.

TABLE 3
titration schedule used in the pardoprunox
group in study S308.2.004

| | | |
|---|---|---|
| Titration | | |
| Step | Total daily dose | Time |

| | | | | |
|---|---|---|---|---|
| 1 | 0.3 | mg | 1 | day |
| 2 | 0.6 | mg | 1 | day |
| 3 | 0.9 | mg | 1 | day |
| 4 | 1.2 | mg | 1 | day |
| 5 | 1.5 | mg | 1 | day |
| 6 | 2.1 | mg | 1 | day |
| 7 | 3.0 | mg | 1 | day |
| 8 | 4.5 | mg | 1 | day |
| 9 | 6.0 | mg | 1 | day |
| 10 | 9.0 | mg | 2 | days |
| 11 | 12.0 | mg | 2 | days |
| 12 | 15.0 | mg | 2 | days |
| 13 | 18.0 | mg | 2 | days |
| 14 | 24.0 | mg | 2 | days |
| 15 | 30.0 | mg | 3-4 | days |
| 16 | 36.0 | mg | 3-4 | days |
| 17 | 45.0 | mg | 3-4 | days |

Approximately 40% of the subjects were treated with pardoprunox doses of ≧30 mg/day from which approximately 16% reached the highest dose level of 45 mg/day, indicating that, while maintaining good tolerability, a subgroup of subjects may need high dosing to obtain adequate efficacy. Further explorative evaluation of the efficacy results between the subgroups of subjects treated with 9-15 mg/day; 18-30 mg/day and >30-45 mg/day and plotting dose/effect data did not provide any clear differences in efficacy. However, interpretation was hampered by the study design using flexible dosing based on tolerability and, in the higher dose range, efficacy.

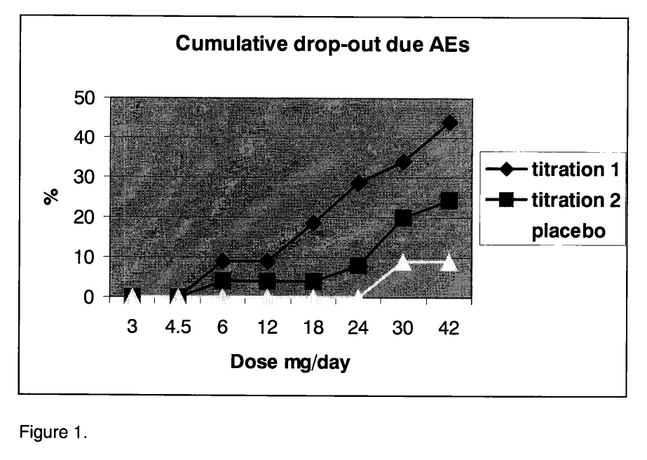

The results of study S308.2.004 indicated that tolerability could possibly be improved by reducing the titration speed, which was applied in the following study S308.3.005. In this study, primarily a safety study, 62 subjects with PD characterized by motor fluctuations were randomized to one of three different treatment arms, consisting of two pardoprunox treatment arms (total daily dose 0.3-42 mg three times daily [tid]; groups 1 [25 subjects] and 2 [26 subjects]) and one placebo arm [11 subjects]). The study included a seven-week ascending dose period of pardoprunox as adjunctive treatment to L-dopa. The effect of two different titration schedules was investigated on the safety and tolerability profile, as well as the need and time required for L-dopa dose and/or regimen adjustments (see Table 4). The difference between the two pardoprunox groups was in titration steps taken to achieve at the same time the main study dose levels, namely Group 1—weekly, larger dose increments versus Group 2—with every 3 and 4-days smaller dose steps to the same ‘end of week’ pardoprunox main doses. In the group with the more gradual titration more than 92% of the subjects (24 out of 26) reached doses of 18 mg/day or higher, and 65% (17 out of 26 subjects) reached the highest dose level of 42 mg/day. The group with the larger dose increments had a much higher drop-out rate (see FIG. 1 depicting the drop out results of the group with larger dose increments (♦), compared with the drop out results of the group with smaller dose increments (▪) and placebo (Δ)).

The drop out rate due to treatment related Adverse Effects was 4% up to the 18 mg/day dose (one subject dropped out of the study at 6 mg/day) in the group with smaller dose increments. Between 18 and 30 mg/day, 15% (four subjects) terminated the study due to treatment related Adverse Effects. There were no particular doses or titration steps that could not be tolerated as defined by causing more than 20% drop outs. The need for L-DOPA adjustment was limited in either treatment arm.

TABLE 4
Titration schedule used in the S308.3.005 study

| | | pardoprunox total | | | |
|---|---|---|---|---|---|
| | | daily dose (mg) | | | |
| | | Group | Group | | |
| Weeks | Days | 1 | 2 | Placebo | |
| | 1 | 0.3 | 0.3 | Placebo | |
| | 2 | 0.6 | 0.6 | Placebo | |
| | 3 | 0.9 | 0.9 | Placebo | |
| | 4 | 1.2 | 1.2 | Placebo | |
| | 5-6 | 1.5 | 1.5 | Placebo | |
| 1 | 7-8 | 2.1 | 2.1 | Placebo | |
| | 9-10 | 3 | 3 | Placebo | |
| | 11-12 | 4.5 | 4.5 | Placebo | |
| 2 | 13-15 | 6 | 6 | Placebo | |
| | 16-18 | 12 | 9 | Placebo | |
| 3 | 19-21 | 12 | 12 | Placebo | Dose level 1 |
| | 22-24 | 18 | 15 | Placebo | |
| 4 | 25-28 | 18 | 18 | Placebo | Dose level 2 |
| | 29-31 | 24 | 21 | Placebo | |
| 5 | 32-35 | 24 | 24 | Placebo | Dose level 3 |
| | 36-38 | 30 | 27 | Placebo | |
| 6 | 39-42 | 30 | 30 | Placebo | Dose level 4 |
| | 43-45 | 42 | 36 | Placebo | |
| 7 | 46-49 | 42 | 42 | Placebo | Dose level 5 |

Based on titration tolerability results of studies S308.2.004 and S308.3.005 the following assumptions were made for designing the titration schedule for the pivotal clinical studies for development of pardoprunox:

doses below 12 mg/day will be tolerated by the vast majority of the patients;

12-24 mg/day will have acceptable tolerability;

30-36 mg/day will be justifiable on an individual patient need basis; and

>45 mg/day is expected to provide only acceptable tolerability for a negligible proportion of patients.

It was anticipated that the minimum efficacious dose was 12 mg/day for the vast majority of patients. However, on an individual basis, as based on the results of studies S308.2.004 and S308.3.005, it was concluded that considerably higher doses of pardoprunox (up to 42 mg/day) may be necessary to achieve efficacy.

This resulted in the following titration schedule which was to be used for the entire pivotal development program.

TABLE 5
Titration scheme used in the pivotal program

| | | Pardoprunox | |
|---|---|---|---|
| | | Daily dose | |
| Weeks | Days | (mg) | |
| | 1 | 0.3 | |
| | 2 | 0.6 | |
| | 3 | 0.9 | |
| | 4 | 1.2 | |
| | 5-6 | 1.5 | |
| 1 | 7-8 | 2.1 | |
| | 9-10 | 3 | |
| | 11-12 | 4.5 | |
| 2 | 13-14 | 6 | |
| | 15 | 6 | |
| | 16-18 | 9 | |
| 3 | 19-21 | 12 | Dose level 1 |
| | 22-24 | 15 | |
| 4 | 25-28 | 18 | Dose level 2 |
| | 29-31 | 21 | |
| 5 | 32-35 | 24 | Dose level 3 |
| | 36-38 | 27 | |
| 6 | 39-42 | 30 | Dose level 4 |
| | 43-45 | 36 | |
| 7 | 46-49 | 42 | Dose level 5 |

In the first pivotal study in early PD patients (study S308.3.001), the efficacy and safety of two fixed dose arms of pardoprunox (6 mg/day [n=115; Group 1] and 12 mg/day [n=118; Group 2]), and a flexible dose arm (12-42 mg/day [n=116; Group 3]) was compared to placebo (n=119) (see Table 6 for titration schedules used). The titration schedule used in Group 3 matches the titration schedule as presented in Table 5.

All active treatment arms were found statistically superior to placebo for the primary outcome measure UPDRS motor score, while tolerability of the 6 mg/day fixed dose group was better than that of the higher dose groups.

TABLE 6
Titration schedule used in study S308.3.001

| | | pardoprunox total daily | | | | |
|---|---|---|---|---|---|---|
| | | dose (mg) | | | | |
| | | Group | Group | Group | | |
| Weeks | Days | 1 | 2 | 3 | Placebo | |
| | 1 | 0.3 | 0.3 | 0.3 | Placebo | |
| | 2 | 0.6 | 0.6 | 0.6 | Placebo | |
| | 3 | 0.9 | 0.9 | 0.9 | Placebo | |
| | 4 | 1.2 | 1.2 | 1.2 | Placebo | |
| | 5-6 | 1.5 | 1.5 | 1.5 | Placebo | |
| 1 | 7-8 | 2.1 | 2.1 | 2.1 | Placebo | |
| | 9-10 | 3 | 3 | 3 | Placebo | |
| | 11-12 | 4.5 | 4.5 | 4.5 | Placebo | |
| 2 | 13-15 | 6 | 6 | 6 | Placebo | |
| | 16-18 | 6 | 9 | 9 | Placebo | |
| 3 | 19-21 | 6 | 12 | 12 | Placebo | Dose level 1 |
| | 22-24 | 6 | 12 | 15 | Placebo | |
| 4 | 25-28 | 6 | 12 | 18 | Placebo | Dose level 2 |
| | 29-31 | 6 | 12 | 21 | Placebo | |
| 5 | 32-35 | 6 | 12 | 24 | Placebo | Dose level 3 |
| | 36-38 | 6 | 12 | 27 | Placebo | |
| 6 | 39-42 | 6 | 12 | 30 | Placebo | Dose level 4 |
| | 43-45 | 6 | 12 | 36 | Placebo | |
| 7 | 46-49 | 6 | 12 | 42 | Placebo | Dose level 5 |

From this study it can be concluded that the minimum effective dose in the early PD patient population is lower than the previously anticipated 12 mg/day in patients with early stage PD and that the doses above 6 mg/day may have a worse tolerability profile.

Based on these data the titration schedule has been modified further to improve the tolerability profile. Two titration schedules will be investigated in patients with early PD with different speed to the dose of 6 mg/day, which is now considered as the maximum dose for early PD patients. The overall speed is reduced by more than 2.5 weeks in the fastest titration scheme (5 weeks up to 6 mg/day with 4 days per dose increment) and by more than 5 weeks for the slowest titration scheme (7 weeks up to 6 mg/day with 6 days per dose increment) as compared to the schedule used in study S308.3.001 (2 weeks). These titration schedules are depicted in Table 7. The doses indicated are daily dosages, divided in three administrations, with the exception of day 0 where a first single dose is given, preferably in the evening.

TABLE 7
Two titration schemes with time segments of 4 and 6 days per dose increment
to a final dose of 1.5, 3 and 6 mg of pardoprunox (Pdx) per day.

| | | Pdx Final | Pdx Final | Pdx Final | Pdx Final | Pdx Final | Pdx Final |
|---|---|---|---|---|---|---|---|
| | | 1.5 mg/d | 3 mg/d | 6 mg/d | 1.5 mg/d | 3 mg/d | 6 mg/d |
| | | Titration 1 | Titration 1 | Titration 1 | Titration 2 | Titration 2 | Titration 2 |
| week | days | (mg/d) | (mg/d) | (mg/d) | (mg/d) | (mg/d) | (mg/d) |
| 1 | 0 | 0.1 | 0.1 | 0.1 | 0.1 | 0.1 | 0.1 |
| | 1 | 0.3 | 0.3 | 0.3 | 0.3 | 0.3 | 0.3 |
| | 2 | 0.3 | 0.3 | 0.3 | 0.3 | 0.3 | 0.3 |
| | 3 | 0.3 | 0.3 | 0.3 | 0.3 | 0.3 | 0.3 |
| | 4 | 0.3 | 0.3 | 0.3 | 0.3 | 0.3 | 0.3 |
| | 5 | 0.3 | 0.3 | 0.3 | 0.6 | 0.6 | 0.6 |
| | 6 | 0.3 | 0.3 | 0.3 | 0.6 | 0.6 | 0.6 |
| 2 | 7 | 0.6 | 0.6 | 0.6 | 0.6 | 0.6 | 0.6 |
| | 8 | 0.6 | 0.6 | 0.6 | 0.6 | 0.6 | 0.6 |
| | 9 | 0.6 | 0.6 | 0.6 | 0.9 | 0.9 | 0.9 |
| | 10 | 0.6 | 0.6 | 0.6 | 0.9 | 0.9 | 0.9 |
| | 11 | 0.6 | 0.6 | 0.6 | 0.9 | 0.9 | 0.9 |
| | 12 | 0.6 | 0.6 | 0.6 | 0.9 | 0.9 | 0.9 |
| | 13 | 0.9 | 0.9 | 0.9 | 1.2 | 1.2 | 1.2 |
| 3 | 14 | 0.9 | 0.9 | 0.9 | 1.2 | 1.2 | 1.2 |
| | 15 | 0.9 | 0.9 | 0.9 | 1.2 | 1.2 | 1.2 |
| | 16 | 0.9 | 0.9 | 0.9 | 1.2 | 1.2 | 1.2 |
| | 17 | 0.9 | 0.9 | 0.9 | 1.5 | 1.5 | 1.5 |
| | 18 | 0.9 | 0.9 | 0.9 | 1.5 | 1.5 | 1.5 |
| | 19 | 1.2 | 1.2 | 1.2 | 1.5 | 1.5 | 1.5 |
| | 20 | 1.2 | 1.2 | 1.2 | 1.5 | 1.5 | 1.5 |
| 4 | 21 | 1.2 | 1.2 | 1.2 | 1.5 | 2.1 | 2.1 |
| | 22 | 1.2 | 1.2 | 1.2 | 1.5 | 2.1 | 2.1 |
| | 23 | 1.2 | 1.2 | 1.2 | 1.5 | 2.1 | 2.1 |
| | 24 | 1.2 | 1.2 | 1.2 | 1.5 | 2.1 | 2.1 |
| | 25 | 1.5 | 1.5 | 1.5 | 1.5 | 3.0 | 3.0 |
| | 26 | 1.5 | 1.5 | 1.5 | 1.5 | 3.0 | 3.0 |
| | 27 | 1.5 | 1.5 | 1.5 | 1.5 | 3.0 | 3.0 |
| 5 | 28 | 1.5 | 1.5 | 1.5 | 1.5 | 3.0 | 3.0 |
| | 29 | 1.5 | 1.5 | 1.5 | 1.5 | 3.0 | 4.5 |
| | 30 | 1.5 | 1.5 | 1.5 | 1.5 | 3.0 | 4.5 |
| | 31 | 1.5 | 2.1 | 2.1 | 1.5 | 3.0 | 4.5 |
| | 32 | 1.5 | 2.1 | 2.1 | 1.5 | 3.0 | 4.5 |
| | 33 | 1.5 | 2.1 | 2.1 | 1.5 | 3.0 | 6.0 |
| | 34 | 1.5 | 2.1 | 2.1 | 1.5 | 3.0 | 6.0 |
| 6 | 35 | 1.5 | 2.1 | 2.1 | 1.5 | 3.0 | 6.0 |
| | 36 | 1.5 | 2.1 | 2.1 | 1.5 | 3.0 | 6.0 |
| | 37 | 1.5 | 3.0 | 3.0 | 1.5 | 3.0 | 6.0 |
| | 38 | 1.5 | 3.0 | 3.0 | 1.5 | 3.0 | 6.0 |
| | 39 | 1.5 | 3.0 | 3.0 | 1.5 | 3.0 | 6.0 |
| | 40 | 1.5 | 3.0 | 3.0 | 1.5 | 3.0 | 6.0 |
| | 41 | 1.5 | 3.0 | 3.0 | 1.5 | 3.0 | 6.0 |
| 7 | 42 | 1.5 | 3.0 | 3.0 | 1.5 | 3.0 | 6.0 |
| | 43 | 1.5 | 3.0 | 4.5 | 1.5 | 3.0 | 6.0 |
| | 44 | 1.5 | 3.0 | 4.5 | 1.5 | 3.0 | 6.0 |
| | 45 | 1.5 | 3.0 | 4.5 | 1.5 | 3.0 | 6.0 |
| | 46 | 1.5 | 3.0 | 4.5 | 1.5 | 3.0 | 6.0 |
| | 47 | 1.5 | 3.0 | 4.5 | 1.5 | 3.0 | 6.0 |
| | 48 | 1.5 | 3.0 | 4.5 | 1.5 | 3.0 | 6.0 |
| | 49 | 1.5 | 3.0 | 6.0 | 1.5 | 3.0 | 6.0 |

With respect to the recognized progression of the disease and foregoing efficacy and tolerability results, doses up to 18 mg/day pardoprunox can be expected to be well-tolerated in the advanced PD population, as 92% of the subjects were able to reach this dose level without tolerability or safety issues in study S308.3.005. Based on the efficacy results of study S308.3.001, the dose of 6 mg/day or lower may already be efficacious, however this may be different in the advanced stage disease population as has been shown for previously developed dopamine agonists. The effective dose range in the advanced PD population is anticipated to be between 3-12 mg/day. For the advanced patient population the fastest of the two titration schedules outlined in Table 9 will probably be used (i.e., 5 weeks up to 6 mg/day). An additional 2 weeks will be added to reach doses up to 12 mg/day (see Table 8).

TABLE 8
Slow titration scheme for advanced PD with time segments
of 4 days per increment to a final dose of 1.5, 3,
6 and 12 mg of pardoprunox (Pdx) per day.

| | | PDX |
|---|---|---|
| week | days | Titration 2 |
| 1 | 0 | 0.1 |
| | 1 | 0.3 |
| | 2 | 0.3 |
| | 3 | 0.3 |
| | 4 | 0.3 |
| | 5 | 0.6 |
| | 6 | 0.6 |
| | 7 | 0.6 |
| 2 | 8 | 0.6 |
| | 9 | 0.9 |
| | 10 | 0.9 |
| | 11 | 0.9 |
| | 12 | 0.9 |
| | 13 | 1.2 |
| | 14 | 1.2 |
| 3 | 15 | 1.2 |
| | 16 | 1.2 |
| | 17 | 1.5 |
| | 18 | 1.5 |
| | 19 | 1.5 |
| | 20 | 1.5 |
| | 21 | 2.1 |
| 4 | 22 | 2.1 |
| | 23 | 2.1 |
| | 24 | 2.1 |
| | 25 | 3.0 |
| | 26 | 3.0 |
| | 27 | 3.0 |
| | 28 | 3.0 |
| 5 | 29 | 4.5 |
| | 30 | 4.5 |
| | 31 | 4.5 |
| | 32 | 4.5 |
| | 33 | 6.0 |
| | 34 | 6.0 |
| | 35 | 6.0 |
| 6 | 36 | 6.0 |
| | 37 | 7.5 |
| | 38 | 7.5 |
| | 39 | 7.5 |
| | 40 | 7.5 |
| | 41 | 9.0 |
| | 42 | 9.0 |
| 7 | 43 | 9.0 |
| | 44 | 9.0 |
| | 45 | 12 |
| | 46 | 12 |
| | 47 | 12 |
| | 48 | 12 |
| | 49 | 12 |

When necessary an additional week can be added to reach doses up to 18 mg/day (See table 9). In this case the dose at day 49 will be 15 mg. In another alternative embodiment the single 0.1 mg dose at day 0 is replaced by three doses of 0.05 mg divided over the day.

TABLE 9
Addition to slow titration scheme for advanced PD
with time segments of 4 days per increment to a final
dose of 18 mg of pardoprunox (Pdx) per day

| | | PDX |
|---|---|---|
| | | Titration |
| week | days | 2 |
| 8 | 50 | 15 |
| | 51 | 15 |
| | 52 | 15 |
| | 53 | 18 |
| | 54 | 18 |
| | 55 | 18 |
| | 56 | 18 |

The titration schedule may be further optimized in terms of simplification of the titration steps/dose increments during the first weeks, up to 3 mg/day. It is expected that it is feasible from a tolerability perspective to delete the steps of 1.2 mg/day and 2.1 mg/day from the above schedule, having eventually simpler steps from 0.9 mg/day onwards which can be considered convenient for dosing instructions in clinical practice (see titration scheme in Table 10).

TABLE 10
Simplified titration scheme

| | | Daily dose |
|---|---|---|
| | Week | (mg) |
| Early and advanced PD | 1 | 0.15 |
| | 2 | 0.3 |
| | 3 | 0.6 |
| | 4 | 1.5 |
| | 5 | 3 |

1. A composition regimen to facilitate the treatment of at least one central nervous system condition, comprising a plurality of unit dosages according to a titration schedule, wherein each of the unit dosages comprises at least one pardoprunox compound, and wherein the unit dosages over the titration schedule increase in amount of the at least one pardoprunox compound to achieve a maintenance dose.

2. The composition regimen according to claim 1, wherein the titration schedule spans at least three time segments.

3. The composition regimen according to claim 2, wherein the titration schedule spans at least five time segments.

4. The composition regimen according to claim 2, wherein the amount of said pardoprunox compound in the unit dosages increases each time segment of the titration schedule.

5. The composition regimen according to claim 2, wherein each time segment is chosen, independent of one another, from one day, two days, three days, four days, five days and seven days.

6. The composition regimen according to claim 2, wherein at least one time segment is at least 2 days.

7. The composition regimen according to claim 1, wherein the unit dosages are given once daily, twice daily or three times daily.

8. The composition regimen according to claim 1, wherein the unit dosages are chosen from single strength dosages of the pardoprunox compound having a strength equivalent to pardoprunox base of from about 0.025 mg to about 0.075 mg, from about 0.075 mg to about 0.125 mg, from about 0.15 mg to about 0.25 mg, from about 0.25 mg to about 0.35 mg, from about 0.35 mg to about 0.45 mg, from about 0.45 mg to about 0.55 mg, from about 0.55 mg to about 0.65 mg, from about 0.65 mg to about 0.8 mg, from about 0.8 mg to about 1.2 mg, from about 1.2 mg to about 1.7 mg, from about 1.7 mg to about 2.2 mg, from about 2.2 mg to about 2.7 mg, from about 2.7 mg to about 3.5 mg, from about 3.5 mg to about 4.5 mg, from about 4.5 mg to about 5.5 mg from about 5.5 mg to about 6.5 mg, from about 6.5 mg to about 7.5 mg, from about 7.5 mg to about 8.5 mg, from about 8.5 mg to about 9.5 mg, from about 9.5 mg to about 11.0 mg, and from about 11.0 mg to about 13.0 mg.

9. The composition regimen according to claim 8, wherein the unit dosages are chosen from single strength dosages of the pardoprunox compound having a strength equivalent to pardoprunox base of from about 0.04 mg to about 0.06 mg, from about 0.08 mg to about 0.12 mg, from about 0.18 mg to about 0.22 mg, from about 0.28 mg to about 0.32 mg, from about 0.38 mg to about 0.42 mg, from about 0.48 mg to about 0.52 mg, from about 0.58 mg to about 0.62 mg, from about 0.68 mg to about 0.72 mg, from about 0.78 mg to about 0.82 mg, from about 0.88 mg to about 0.92 mg, from about 0.98 mg to about 1.1 mg, from about 1.3 to about 1.7 mg, from about 1.8 mg to about 2.2 mg, from about 2.3 mg to about 2.7 mg, from about 2.8 mg to about 3.2 mg, from about 3.8 to about 4.2 mg, from about 4.8 to about 5.2 mg, from about 5.8 to about 6.2 mg, from about 6.8 mg to about 7.2 mg, from about 7.8 mg to about 8.2 mg, from about 8.8 mg to about 9.2 mg, from about 9.8 mg to about 10.4 mg, and from about 11.6 mg to about 12.4 mg.

10. The composition regimen according to claim 9, wherein the unit dosages are chosen from single strengths doses of the pardoprunox compound having a strength equivalent to pardoprunox base of about 0.05 mg about 0.1 mg, about 0.2 mg, about 0.3 mg, about 0.4 mg, about 0.5 mg, about 0.6 mg, about 0.7 mg, about 1.0 mg, about 1.5 mg, about 2.0 mg, about 2.5 mg, about 3.0 mg, about 4.0 mg, about 5.0 mg, about 6.0 mg, about 7.0 mg, about 8.0 mg, about 9.0 mg, about 10.0 mg, and about 12.0 mg.

11. The composition regimen according to claim 2, wherein, following initial administration of a first dosage of the pardoprunox compound in the first time segment, the strength of the dosage of pardoprunox compound of the following time segment is about 1.1 to 3 times the dosage of the preceding time segment.

12. The composition regimen according to claim 1, wherein said pardoprunox compound is pardoprunox hydrochloride.

13. A kit comprising at least three different compositions containing increasing amounts of at least one pardoprunox compound.

14. The kit according to claim 13, comprising at least seven different compositions containing increasing amounts of a pardoprunox compound.

15. The kit according to claim 13, wherein the unit dosages are chosen from single strength doses of a pardoprunox compound having a strength equivalent to pardoprunox base of from about 0.025 mg to about 0.075 mg, from about 0.075 mg to about 0.15 mg, from about 0.15 mg to about 0.25 mg, from about 0.25 mg to about 0.35 mg, from about 0.35 mg to about 0.45 mg, from about 0.45 mg to about 0.55 mg, from about 0.55 mg to about 0.65 mg, from about 0.65 mg to about 0.8 mg, from about 0.8 mg to about 1.2 mg, from about 1.2 mg to about 1.7 mg, from about 1.7 mg to about 2.2 mg from about 2.2 mg to about 2.7 mg, from about 2.7 mg to about 3.5 mg, from about 3.5 mg to about 4.5 mg, from about 4.5 mg to about 5.5 mg from about 5.5 mg to about 6.5 mg, from about 6.5 mg to about 7.5 mg, from about 7.5 mg to about 8.5 mg, from about 8.5 mg to about 9.5 mg, from about 9.5 mg to about 11.0 mg and from about 11.0 mg to about 13.0 mg.

16. The kit according to claim 15, wherein the unit dosages are chosen from single strength doses of a pardoprunox compound having a strength equivalent to pardoprunox base of from about 0.04 mg to about 0.06 mg, from about 0.08 mg to about 0.12 mg, from about 0.18 mg to about 0.22 mg, from about 0.28 mg to about 0.32 mg, from about 0.38 mg to about 0.42 mg, from about 0.48 mg to about 0.52 mg, from about 0.58 mg to about 0.62 mg, from about 0.68 mg to about 0.72 mg, from about 0.78 mg to about 0.82 mg, from about 0.88 mg to about 0.92 mg, from about 0.98 mg to about 1.1 mg, from about 1.3 to about 1.7 mg, from about 1.8 mg to about 2.2 mg, from about 2.3 mg to about 2.7 mg, from about 2.8 mg to about 3.2 mg, from about 3.8 to about 4.2 mg, from about 4.8 to about 5.2 mg, from about 5.8 to about 6.2 mg, from about 6.8 mg to about 7.2 mg, from about 7.8 mg to about 8.2 mg, from about 8.8 mg to about 9.2 mg, from about 9.8 mg to about 10.4 mg, from about, and from about 11.6 mg to about 12.4 mg.

17. The kit according to claim 16, wherein the unit dosages are chosen from single strength doses of a pardoprunox compound having a strength equivalent to pardoprunox base of about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.5 mg, about 2.0 mg about, about 2.5 mg, about 3.0 mg, about 4.0 mg, about 5.0 mg, about 6.0 mg, about 7.0 mg, about 8.0 mg, about 9.0 mg, about 10.0 mg, and about 12.0 mg.

18. The kit according to claim 17, wherein the unit dosages are chosen from single strength doses of a pardoprunox compound having a strength equivalent to pardoprunox base of about 0.1 mg, about 0.2 mg, about 0.3 mg, about 0.4 mg, about 0.5 mg, about 0.7 mg about, about 1.0 mg, about 1.5 mg, about 2.0 mg, about 2.5 mg, and about 3.0 mg.

19. The kit according to claim 18, wherein the unit dosages are chosen from single strength doses of a pardoprunox compound having a strength equivalent to pardoprunox base of about 0.05 mg, about 0.1 mg, about 0.2 mg, about 0.5 mg, and about 1.0 mg.

20. The kit according to claim 13, wherein the strength of the dosage of said pardoprunox compound of any strength, with the exception of the lowest strength, is about 1.1 to 3 times the dosage of the preceding strength.

21. The kit according to claim 20, wherein the strength of the dosage of said pardoprunox compound of any strength, with the exception of the lowest strength, is 1.5 to 2.5 times the dosage of the preceding strength.

22. The kit according to claim 13, wherein said pardoprunox compound is pardoprunox hydrochloride.

23. The kit according to claim 13, in the form of a blister package or a dispenser.

24. A method for treating a patient suffering from Parkinson's disease or Restless leg Syndrome comprising:
initiating treatment with a composition regimen comprising at least three unit dosages of a composition according to a titration schedule, each of the unit dosages comprises at least one pardoprunox compound, wherein the unit dosages over the entire titration schedule increase in an amount of the at least one pardoprunox compound; and
maintaining treatment with administration of a maintenance dose of the at least one pardoprunox compound.

25. The method according to claim 24, wherein the maintenance dose ranges from 0.9 mg/day up to 42 mg/day.

26. A method for treating a patient suffering from Parkinson's disease or Restless leg Syndrome comprising:
initiating treatment with a composition regimen comprising at least three unit dosages of a composition according to a titration schedule, wherein each of the unit dosages comprises at least one pardoprunox compound, wherein the unit dosages over the entire titration schedule increase in an amount of the at least one pardoprunox compound, wherein the titration schedule spans at least three time segments, wherein the unit dosages are chosen from single strength doses of a pardoprunox compound having a strength equivalent to pardoprunox base of from about 0.025 mg to about 0.075 mg, from about 0.075 mg to about 0.15 mg, from about 0.15 mg to about 0.25 mg, from about 0.25 mg to about 0.35 mg, from about 0.35 mg to about 0.45 mg, from about 0.45 mg to about 0.55 mg, from about 0.55 mg to about 0.65 mg, from about 0.65 mg to about 0.8 mg, from about 0.8 mg to about 1.2 mg, from about 1.2 mg to about 1.7 mg, from about 1.7 mg to about 2.2 mg from about 2.2 mg to about 2.7 mg, from about 2.7 mg to about 3.5 mg, from about 3.5 mg to about 4.5 mg, from about 4.5 mg to about 5.5 mg from about 5.5 mg to about 6.5 mg, from about 6.5 mg to about 7.5 mg, from about 7.5 mg to about 8.5 mg, from about 8.5 mg to about 9.5 mg, from about 9.5 mg to about 11.0 mg, and from about 11.0 mg to about 13.0 mg.
and
maintaining treatment with administration of a maintenance dose of the at least one pardoprunox compound.

27. The method according to claim 26, wherein the maintenance dose ranges from 0.9 mg/day up to 42 mg/day.

28. A method for treating a patient suffering from Parkinson's Disease or Restless Leg Syndrome comprising:
initiating treatment with a composition regimen comprising at least three unit dosages of a composition according to a titration schedule, wherein each of the unit dosages comprises at least one pardoprunox compound, wherein the unit dosages over the entire titration schedule increase in an amount of the at least one pardoprunox compound, wherein the initiation treatment diminishes side effects associated with administration of the at least one pardoprunox compound without the titration schedule; and
maintaining treatment with administration of a maintenance dose of the at least one pardoprunox compound.

29. The method according to claim 28 wherein the maintenance dose ranges from 0.9 mg/day up to 42 mg/day.
